(19) United States
(12) Patent Application Publication
(10) Pub. No.: US 2011/0136921 A1
(43) Pub. Date: Jun. 9, 2011

(54) SUSTAINED RELEASE COMPOSITION

(76) Inventors: Nilesh Tanhaji Dumbre, Maharashtra (IN); Amelia Makarand Avachat, Maharashtra (IN); Nandu Deorkar, Cedar Knolls, NJ (US); James Farina, Nazareth, PA (US); Liliana Miinea, Whitehall, PA (US)

(21) Appl. No.: 12/737,663

(22) PCT Filed: Aug. 6, 2009
(86) PCT No.: PCT/US09/52956

§ 371 Date: Feb. 4, 2011

(30) Foreign Application Priority Data
Aug. 7, 2008 (IN) 1676/MUM/2008

(51) Int. Cl. A61K 47/26; B29B 9/12; B29B 9/10

(52) U.S. Cl. 514/780; 514/777; 264/12; 264/6

(57) ABSTRACT

A sustained release composition comprising spray dried particles of at least one polysaccharide gum and at least one polyhydric sugar alcohol, as well as methods of making the sustained released composition are provided. A sustained release pharmaceutical solid dosage form, and a method of making the solid dosage form by compression are also provided.

### BACKGROUND OF INVENTION

Sustained release compositions allow administration of an effective dose of a drug over an extended period of time. Sustained release is advantageous since patient's side effects arising out of administering an immediate release therapy may be reduced. Sustained or prolonged-release dosage forms of various drugs are known in the art. Conventional sustained release dosage forms include the use of a polymer matrix, as well as complexing the drug with an ion exchange resin forming a drug-ion exchange resin complex particle. After administration, the drug is slowly released from the complex or matrix over time, thereby providing a continuous delivery of drug to the patient. Conventional pharmaceutical sustained release compositions often include polymers such as hydroxylproplyl methylcellulose, sodium carboxy methylcellulose, hydroxylpropyl cellulose, methyl cellulose, chitosan, and natural gums to sustain drug delivery.

Polysaccharide gums, for example guar gum, locust bean gum, xanthan gum, karaya gum, tara gum and Konjac gum are known to be potential hydrophilic matrix carriers for sustained delivery of drugs with varying solubility. In pharmaceutical formulations, guar gum has been used as a binder, disintegrant, suspending agent, thickening agent and stabilizing agent as well as a carrier in colon targeting delivery system. It is practically insoluble in organic solvents; in hot or cold water it disperses and swells almost immediately to form a highly viscous thixotropic solution. Viscosity is dependent on temperature, time, concentration, pH, rate of agitation and particle size. Prolonged heating reduces viscosity. Guar gum is found to have poor flow properties, poor compressibility and uneven particle size and is to be incorporated in the matrix tablets in large proportion (30 to 90%), and tablets containing guar gum are typically prepared by wet granulation technique. While guar gum is a well accepted pharmaceutical excipient used in low proportions as a binder, disintegrant or carrier in conventional dosage forms, it is not a preferred excipient for materials that can be directly compressed.

Prior art discloses the use of guar gum in a tricalcium phosphate agglomerate formed by spray drying an aqueous slurry of tricalcium phosphate and a binder which may be guar gum to enable direct compression of a chewable oral dosage form. Guar gum has also been used in a method for stabilizing proteins where an aqueous solution of the protein and an aqueous polysaccharide gum such as guar gum are spray dried or lyophilized and then coated and encapsulated. Another method utilizing guar gum is a method of making a solid interpolymer complex for use as a controlled release matrix for oral administration, from a first polymer and one or more second complementary polymers capable of complexing with the first polymer to form the interpolymer complex, wherein one of the polymers is guar gum and the process comprises several steps including a step of spray drying to remove the solvent.

The prior art also discloses compositions containing heteropolyschaarides such as xanthan gum and locust bean gum, cross-linked along with an inert diluent prepared by a wet granulation process. This method therefore requires the use of two polysaccharide gums and a wet granulation process.

The composition of the present invention is prepared by spray drying. Spray drying is a commonly used, rapid, continuous method of drying a liquid feed through a hot gas that eliminates additional processing for obtaining dry material. It is essentially a three-step drying process consisting of: (1) atomization of a liquid feed into a spray of fine droplets; (2) suspension of droplets by a heated gas stream, evaporation of the liquid; and (3) separation of the dried powder from the gas stream and collection of same.

The process of spray drying and other drying processes such as freeze drying are applied widely for obtaining dried products, but there exists no prior art for the application of such processes to improve the properties of polysaccharide gums.

### SUMMARY OF INVENTION

In an illustrative aspect of the present invention there is provided a sustained release composition comprising substantially spherical particles of at least one polysaccharide gum in combination with at least one polyhydric sugar alcohol.

In another illustrative aspect of the present invention there is provided a sustained release composition comprising a spray dried mixture of at least one polysaccharide gum in combination with at least one polyhydric sugar alcohol.

In yet another illustrative aspect of the present invention there is provided a method for producing a sustained release composition, the method comprising dissolving at least one polysaccharide gum and at least one polyhydric sugar alcohol in a solvent to form a solution/suspension, and spray drying the solution/suspension to form particles of the sustained release composition.

In still another illustrative aspect of the present invention there is provided method of making a sustained release pharmaceutical solid dosage form, the method comprising dissolving at least one polysaccharide gum and at least one polyhydric sugar alcohol in a solvent to form a solution/suspension; spray drying the solution/suspension to form particles of a sustained release composition; mixing the sustained release composition with at least one filler and at least one active pharmaceutical ingredient to form a tabletting mixture; and compressing the tabletting mixture to form the sustained release pharmaceutical dosage form.

In a further illustrative aspect of the present invention there is provided a sustained release pharmaceutical solid dosage form comprising a spray dried mixture of at least one polysaccharide gum in combination with at least one polyhydric sugar alcohol; at least one filler; and at least one active pharmaceutical ingredient.

In another illustrative aspect of the present invention there is provided a sustained release composition comprising a spray dried mixture of at least one polysaccharide gum in combination with at least one oligosaccharide.

In yet another illustrative aspect of the present invention there is provided a method for producing a sustained release composition, the method comprising mixing at least one polysaccharide gum and at least one oligosaccharide in a solvent to form a solution/suspension, and spray drying the solution/suspension to form particles of the sustained release composition. A solid dosage form may be produced from these particles by mixing the sustained release composition with at least one filler and at least on active pharmaceutical ingredient to form a tabletting mixture, and compressing the tabletting mixture to form the sustained release pharmaceutical dosage form.

In still another illustrative aspect of the present invention there is provided a sustained release pharmaceutical solid dosage form comprising a spray dried mixture of at least one polysaccharide gum in combination with at least one oligosaccharide; at least one filler; and at least one active pharmaceutical ingredient.

In a further illustrative aspect of the present invention there is provided a sustained release composition comprising a spray dried mixture of at least one polysaccharide gum in combination with at least one polyhydric sugar alcohol and at least one oligosaccharide.

### BRIEF DESCRIPTION OF DRAWINGS

FIG. 1 is an illustration of SEM micrographs of guar gum.

FIG. 2 is an illustration of SEM micrographs of mannitol (Pearlitol 160 C—Roquette).

FIG. 3 is an illustration of SEM micrographs of spray dried guar gum/mannitol, 1:1 according to Example 1.

FIG. 4 is an illustration of SEM micrographs of spray-cried guar gum/mannitol, 1:4 according to Example 14.

FIG. 5 is an illustration of an SEM micrograph of locust bean gum (cold water soluble).

FIG. 6 is an illustration of SEM micrographs of locust bean gum (cold water soluble): mannitol, 1:1, according to Example 15.

FIG. 7 is an illustration of SEM micrographs of inulin (Orafti ST Gel).

FIG. 8 is an illustration of SEM micrographs of spray dried guar gum/inulin according to Example 16.

FIG. 9 is a dissolution profile of diclofenac sodium formulations F1-F4 according to Example 6.

FIG. 10 is a dissolution profile of diclofenac sodium formulations F5-F7 and the marketed drug, Voveran SR, according to Example 6.

FIG. 11 is a dissolution profile of Venlafaxine HCL according to Example 7.

FIG. 12 is a dissolution profile of Guaifenesine Tablets according to Example 8.

FIG. 13 is a dissolution profile of tramadol hydrochloride according to Example 9.

FIG. 14 is a dissolution profile of diclofenac sodium formulations according to Example 10, through 24 hours.

FIG. 15 is a dissolution profile of diclofenac sodium formulations according to Example 10, through 8 hours.

FIG. 16 is a dissolution profile of acetaminophen formulations according to Example 11.

### DETAILED DESCRIPTION

The present invention provides improved sustained release pharmaceutical compositions comprising polysaccharide gums and polyhydric sugar alcohols. More particularly, the invention provides a novel spray dried sustained release composition comprising polysaccharides gums such as guar gum, locust bean gum, xanthan gum, karaya gum tam gum or Konjac gum in combination with polyhydric sugar alcohol. The composition provides enhanced flow properties, uniform spherical particle and release retardant properties for the formulation of novel drug delivery systems.

It has been unexpectedly discovered that a composition produced by spray drying a solution/suspension including at least one polysaccharide gum and at least one polyhydric sugar alcohol results in a product that provides a sustained release profile when formulated with an API. Physical mixing or wet granulation of polysaccharide gum and polyhydric sugar alcohol components does not provide a composition suitable for sustained release applications, although a limited release retardation may be observed.

Polysaccharide gums are either hydrophobic or hydrophilic high molecular weight molecules that produce gels or high viscosity solutions with a low level of the gum present. Suitable polysaccharide gums for the present invention include guar gum, xanthan gum, locust bean gum, karaya gum, tara gum, Konjac gum and mixtures thereof. Guar Gum is obtained from the seed of the legume Cyamopsis tetragonolobus. Guar gum forms a solution/suspension at 1% with a high viscosity of 5600 CPS. The solution/suspension is non-Newtonian and the viscosity changes with temperature, at 85° C. a 1% solution/suspension has a viscosity of about 2500 CPS. Guar gum is more soluble than locust bean gum and is not self gelling.

Locust bean gum is obtained from the seed of the carob tree. Locust bean gum forms a solution/suspension at 1% with a viscosity of 3000 CPS. Locust bean gum is only slightly soluble in water and must be heated to 85° C. to achieve full viscosity. Locust bean gum in not self gelling. Gum Karaya is exuded from Sterculia urens a large bushy tree. Karaya gum forms a solution/suspension at 1% with a viscosity of 1000 CPS. Karaya is one of the least soluble gums and usually forms a uniform dispersion.

In accordance with the present invention, spray drying of solution/suspensions of polysaccharide gum in the range of 0.25%-1.0% of solid content was attempted. The viscosity of the solution/suspensions were in the range of 350-4800 cp, making spray drying of polysaccharide gum solution/suspensions alone impractical, as the polysaccharide gum stuck to the wall of drying chamber.

It was surprisingly determined that a combination of polysaccharide gum with a sugar improved the spray characteristics of the polysaccharide gum. The polysaccharide gum was combined in various proportions with at least one polyhydric sugar alcohol selected from mannitol, xylitol, maltitol, lactitol, sorbitol, erythritol, isomalt and mixtures thereof. The combination reduced the viscosity of the polysaccharide gum adequately to result in excellent spray characteristics and ease in spray drying, resulting in spray dried polysaccharide. In the illustrative examples given herein, the polysaccharide gum and polyhydric sugar gum were physically mixed prior to adding a liquid to form a solution/suspension. However, it is noted that this step is not required, and further that it is not required that the components be mixed together in any particular order.

In an illustrative, non-limiting embodiment, the polysaccharide gum: polyhydric sugar alcohol ratio is typically about 1:0.5 to 1:10, with a presently preferred ratio of about 1:1 to 1:3. The polyhydric sugar alcohols being non-hygroscopic, they were combined effectively with moisture sensitive ingredients as well. Further, the polyhydric sugar alcohol prevented thickening of the aqueous dispersion and also increased the hydrophobicity of the polysaccharide gum/polyhydric sugar alcohol material.

Most surprisingly, the co-processed, spray dried forms of the polysaccharide gum/polyhydric sugar alcohol of the instant invention are suitable for direct compression, and result in a sustained release solid dosage form. In an alternate embodiment, the spray dried particles may be a preferred excipient for wet granulation as well.

The spray drying processes used are conventional processes known in the art. In one illustrative embodiment, the polysaccharide gum and polyhydric sugar alcohol solution/suspension was sprayed into spray drier at a feed rate of 45-150 ml/hour. The inlet and outlet temperatures varied from 100-220° and 60-125° C. respectively. The atomizing air pressure varied from 1-4 bars, the compressed air flow was 45-85% and vacuum was 70-300 mm. The process yield varied from 20-60%. Examples 1 and 10 are non-limiting illustration of the production of guar gum/mannitol spray dried particles of the present invention.

As clearly shown in Example 10, the spray dried polysaccharide gum/polyhydric sugar alcohol particles of the present invention produce an exceptional sustained release dissolution profile, as compared to the dissolution profile of tablets produced from polysaccharide gum and polyhydric sugar alcohol that were merely physically mixed.

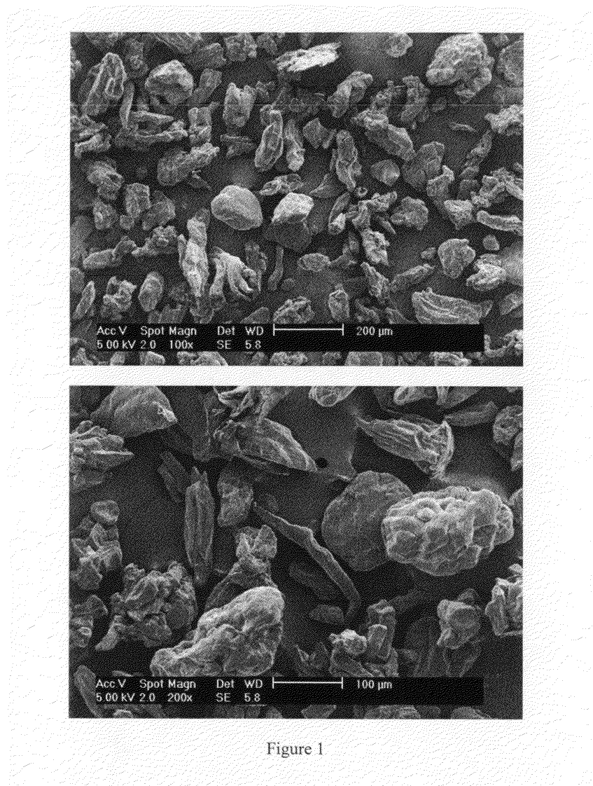

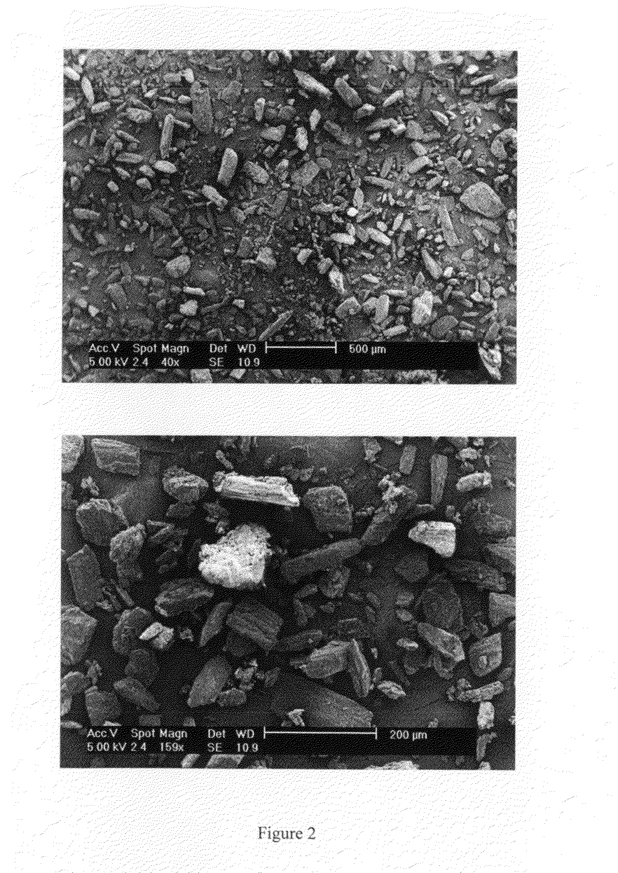

The powder morphology, the shape and surface topography of plain guar gum, mannitol, and the spray dried polysaccharide gum/polyhydric sugar alcohol particles, were observed by scanning electron microscopy (SEM). SEM micrographs of guar gum, shown in FIG. 1 showed its polygonal shape with porous surface, while SEM of mannitol, shown in FIG. 2 showed smooth surface without any porous structure. It is noted the term ‘plain’ defines commercially available composition prior to spray drying.

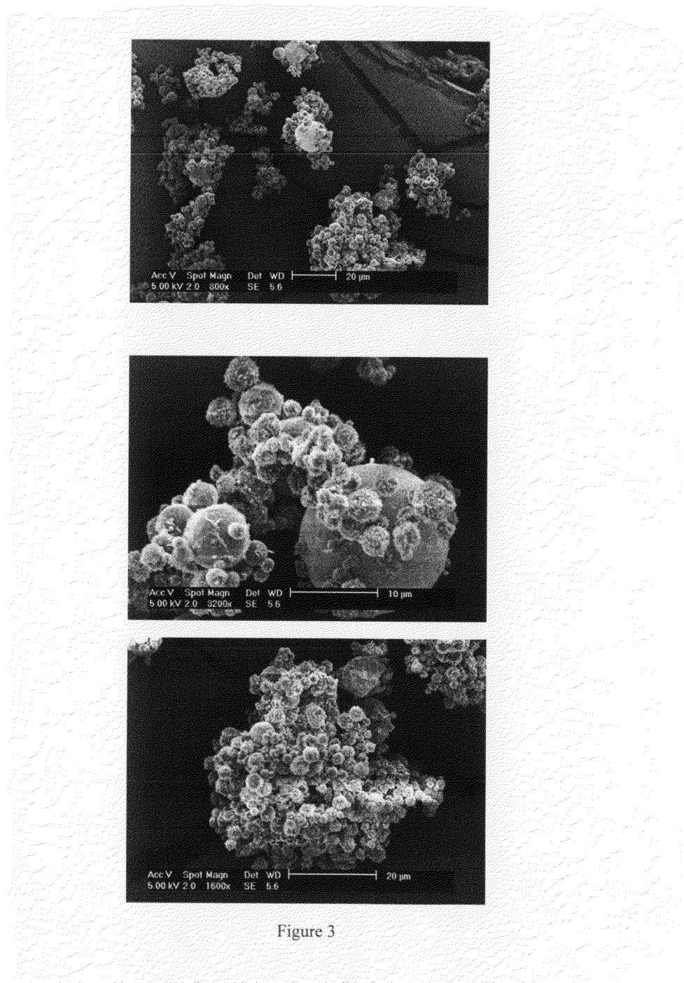

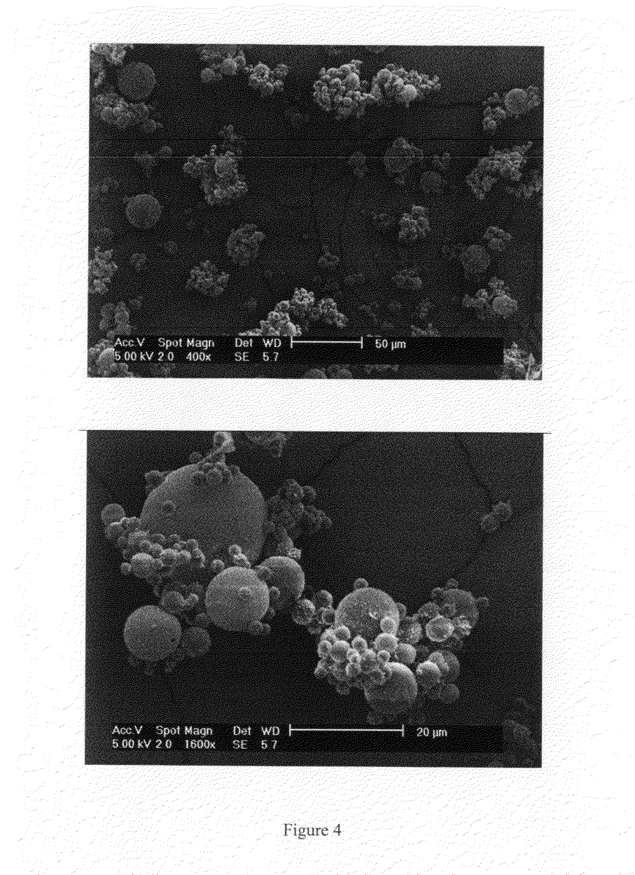

The spray dried particles of guar gum with mannitol were evaluated for powder morphology, powder characteristics and possible interactions between gums and sugars and exemplified herein. The spray dried polysaccharide gum/polyhydric sugar alcohol particles were found to be spherical, with smaller particle size than gum as such with favorable angle of repose and Carr's index. The spray dried polysaccharide gum/polyhydric sugar alcohol particles were substantially spherical in shape with rough surface without any porous structure and were free flowing, as shown in FIGS. 3 and 4, according to Examples 1 and 14, respectively.

DSC and FTIR analysis of the starting materials, guar gum and mannitol, as well as particles of a physical mixture of guar gum and mannitol, and spray dried polysaccharide gum/polyhydric sugar alcohol particles according to Example 1 revealed no reaction between the starting materials, and also showed loss of bound form of water present in guar gum. (See Examples 4 and 5.)

The spray dried particles of the instant invention were used to formulate drug dosage forms. The spray dried particles were further formulated as release retardant agents in novel drug delivery systems as exemplified herein.

Sustained release dosage forms utilizing the spray dried polysaccharide gum/polyhydric sugar alcohol particles were prepared with both highly soluble active pharmaceutical ingredients (API), such as tramadol hydrochloride (Example 9) and venlafaxine hydrochloride (Example 7) and sparingly soluble API such as guaiphenesin (Example 8) and diclofenac sodium (Examples 6 and 10.) It has therefore been clearly illustrated that the spray dried particles of the present invention are suitable for a wide variety of API. Typically, the sustained release formulation of the present invention will be mixed with a filler and the API prior to compression to produce the solid dosage form. Selection of a filler compatible with the specific API, as is well known in the art, places little if any limitation on the number and types of API which can be utilized with the present invention. Suitable fillers for use with the present invention are well known in the art, and include but are not limited to microcrystalline cellulose (MCC), lactose, dicalcium phosphate and mixtures thereof.

In an alternate embodiment, the spray dried particles of the present invention may be mixed with a conventional filler, for example hydroxypropyl methylcellulose (HPMC), polyvinylpyrrolidone (PVP), starch and mixtures thereof, and at least one API for wet granulation.

The dosage forms were formulated with the spray dried particles ranging from 5% to 60% of the formulation. The higher percentages of the spray dried particles were for drugs that have more solubility whereas the spray dried compressed material was exercised in lower quantities in drugs with poor solubility. But the release profiles of the drugs were sustained with the co-processed material of polysaccharide and sugar of the instant invention, independent of the solubility of the drug. Additional ingredients in the formulations, such as pharmaceutically acceptable excipients including filler and lubricants, may be utilized with the present invention as is well known in the art. The tablets were evaluated for physical parameters and the dissolution profile and compared with that of marketed formulations and formulation prepared with conventionally accepted release retardants.

The processing of the polysaccharide gum in accordance with the invention made it more flowable, spherical, and uniform in particle size and most importantly imparted release retardant properties as exemplified herein. This provided a ready-to-use, simple sustained release excipient with wide ranging applications in formulation development without the disadvantages of batch-to-batch non-uniformity found in naturally sourced excipients. Further, newly synthesized polymers need to be approved by regulatory authorities before being available for use. The polysaccharides of the invention being well accepted excipients are only undergoing a process of spray drying and this does not change their regulatory status as pharmaceutically accepted excipients and are generally regarded as safe with practically no adverse reports.

The spray dried particles of the present invention find application in conventional dosage forms such as tablets, capsules and granules. These particles are especially well suited for use as sustained release, extended release or delayed release, colon targeted and gastro retentive dosage forms.

In an alternate embodiment the sustained release composition can also be made by spray drying a polysaccharide gum and a mixture of oligo- and poly-saccharides which are composed of fructose units linked together by β(1-2) linkages. Almost every molecule of the mixture of oligo- and poly-saccharides which are composed of fructose units linked together by β(1-2) linkages is terminated by a glucose unit. The total number of fructose and glucose units (degree of polymerization) of the oligo- and polysaccharide which are composed of fructose units linked together by β(1-2) linkages ranges mainly between 3 to 60. A relevant example of the class of materials that are composed of a mixture of oligo- and polyfructose as described above is chicory inulin.

We have surprisingly discovered that combining a polysaccharide gum and chicory inulin in solution/dispersion allows the easy spray drying of a polysaccharide gum, and that the resulting spray dried polysaccharide gum/inulin material has the property of retardation of drug release.

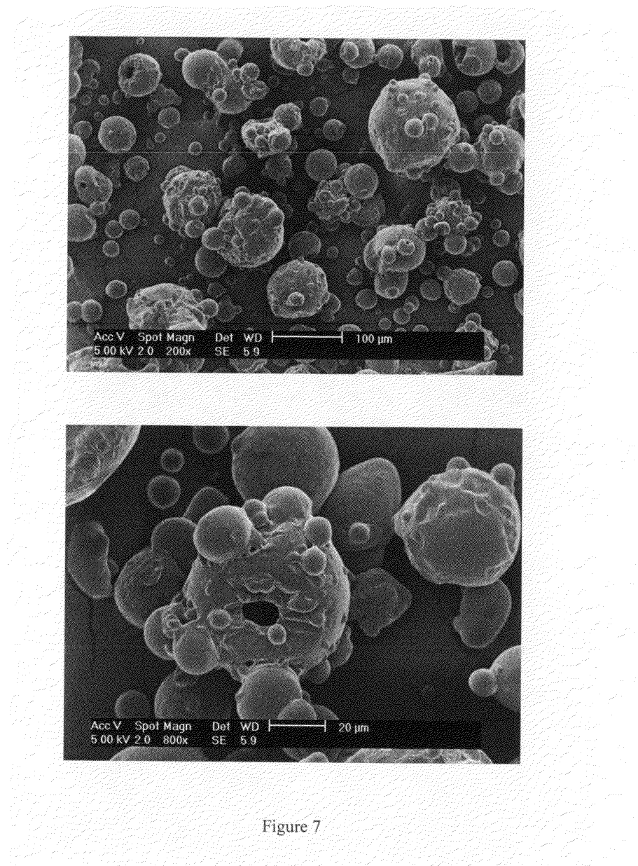

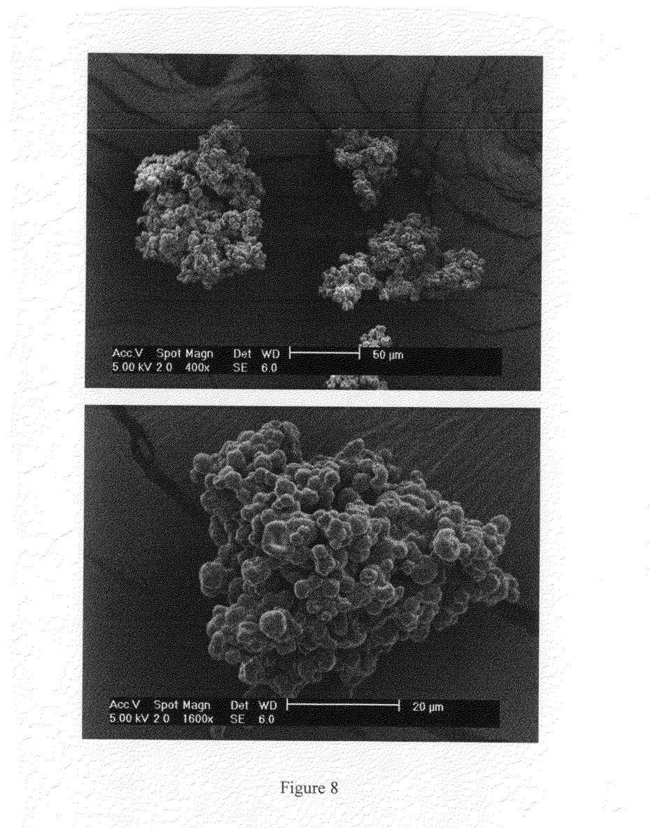

Inulin (also known as oligofructose, polyfructose) is a naturally occurring polysaccharide consisting of a linear chain of linked D-fructose molecules having one terminal glucose molecule of the general formula: C₆F₁₁O₄(C₆H₁₁O₄)ₙOH, with a mol weight of up to 5000. Grades of inulin that are obtained by partial enzymatic hydrolysis of “chicory inulin,” consisting of oligofructose with a degree of polymerization between 2 and 8 are also suitable for the present invention. SEM micrographs of plain inulin and spray dried inulin/guar gum according to Example 16 are shown in FIGS. 7 and 8 respectively.

In another alternate embodiment of the present invention, the at least one polysaccharide gum may be mixed with a combination of at least one polyhydric sugar alcohol and at least one oligosaccharide in a solvent to form a spray dryable solution/suspension. The resulting spray dried particles provide an improved sustained release material. The polysaccharide gum/polyhydric sugar alcohol/oligosaccharide spray dried particles are suitable for use in the methods and dosage forms discussed herein relating to the polysaccharide gum/polyhydric sugar alcohol spray dried particles.

The following Examples are provided for illustrative purposes only and are not limiting of the present invention disclosed and claimed herein.

### EXAMPLE 1

Preparation of Spray Dried Particles:

Spray drying of a solution/suspension of mannitol with guar gum was performed using a spray dryer. Spray dried material 1 was guar gum:mannitol in the ratio of 1:1 and spray dried material 2 was guar gum:mannitol in the ratio of 1:2. The solution/suspension was fed through the nozzle (diameter 0.7mm) at the top of the drying chamber of spray dryer by means of peristaltic pump. The spray dryer operated in co-current airflow. The feed rate varied between 50-200 ml/hr, at an inlet drying temperature of 100-150° C. and outlet drying temperature of 60-100° C. The atomizing air pressure was 1-3 bar and compressed air flow varied between 60-300 mmWc. The spray dried particles were collected in a reservoir attached to cyclone, cooled down to room temperature, sieved and stored in sealed vials.

### EXAMPLE 2

The powder morphology, the shape and surface topography of plain guar gum, plain mannitol, and the spray dried material according to Example 1, were observed by scanning electron microscopy (SEM), shown in FIGS. 1, 2 and 3 respectively. SEM micrographs of guar gum showed its polygonal shape with porous surface, while mannitol showed smooth surface without any porous structure. Spray dried materials were almost spherical in shape with rough surface without any porous structure and was free flowing.

### EXAMPLE 3

The powder characteristics such as angle of repose were determined by fixed funnel and standing cone method. Bulk density and true density, and Carr's index were also determined.

TABLE 1

| | | Spray dried | Spray dried |
|---|---|---|---|
| Powder characteristics | Guar Gum | material 1 | material 2 |
| Shape | Irregular | Spherical | Spherical |
| Size distribution, μm | 30-100 | 1-20 | 1-20 |
| Angle of Repose | 45 | 22.27 | 27.34 |

### EXAMPLE 4

The possibility of any interaction between guar gum and mannitol during spray drying, and between spray dried material and drug was assessed by carrying out thermal analysis on plain guar gum, plain mannitol, physical mixture of guar gum, spray dried material and tablet matrix blend using DSC. DSC analysis reveals that there is no reaction between guar gum and mannitol during spray drying and it also shows loss of bound form of water that was present in guar gum.

### EXAMPLE 5

The Fourier transform infrared (FTIR) spectroscopy of plain guar gum, plain mannitol, physical mixture of guar gum and mannitol, and spray dried material were conducted by scanning in the wavelength range of 400-4000 cm⁻¹. No change in nature of gum and sugar was observed.

### EXAMPLE 6

Tablets of Diclofenac Na were manufactured using different concentrations of the co-processed spray dried material consisting of guar gum and mannitol. Hardness of tablets ranged from 6-7 kg/cm².

Tablets were also prepared by using physical mixture of guar gum as such and mannitol to show the effect of co-processed material on the release of drug from tablet. Tablets were also prepared by using HPMC to compare the release properties of spray dried material with HPMC. Tablets were prepared by blending weighed amount of diclofenac Na, and the corresponding excipients as shown in Table 2.

TABLE 2
Formulation of Diclofenac Na. (Quantity per Tablet in mg)

| Ingredients | F1 | F2 | F3 | F4 | F5 | F6 | F7 |
|---|---|---|---|---|---|---|---|
| Diclofenac Na | 100 | 100 | 100 | 100 | 100 | 100 | 100 |
| MCC 102 | 100 | 100 | 100 | 100 | 100 | 100 | 100 |
| HPMC K100M | — | — | — | — | — | 26 | — |
| SDGGMN 1 | 24 | 30 | — | — | — | — | 26 |
| SDGGMN 2 | — | — | 45 | 30 | | — | — |
| PMGGMN 1 | — | — | — | — | 26 | — | — |
| Mg Stearate | 2 | 2 | 2 | 2 | 2 | 2 | 2 |
| Talc | 4 | 4 | 4 | 4 | 4 | 4 | 4 |
| Total | 230 | 236 | 251 | 236 | 232 | 232 | 232 |
| % of Release | 10.43 | 12.71 | 17.92 | 12.71 | 10.43 | 10.43 | 10.43 |
| retardant material | | | | | | | |

F—Formulations, MCC102—Microcrystalline Cellulose, HPMC K100M—Hydroxypropylmethylcellulose (1,00,000 cp), SDGGMN1—spray dried guar gum and mannitol(1:1), SDGGMN2—spray dried guar gum and mannitol(1:2), PMGGMN 1—physical mixture of guar gum and mannitol(1:1),

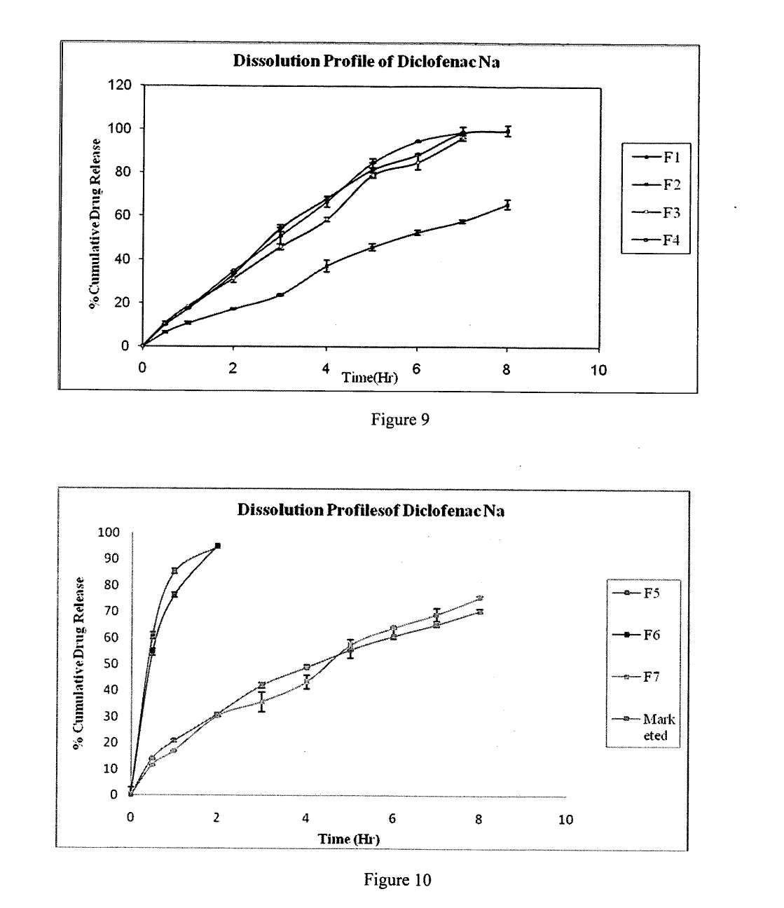

The tablets were evaluated for typical physical tabletting parameters and for dissolution. The dissolution results are graphically represented herein in FIG. 9 and FIG. 10. The comparative profile of dissolution of co-processed material of gum with sugar and that of the marketed sustained release tablet, Voveran SR, validates the claim of spray dried polysaccharide as a release retardant material. Formulation containing physical mixture of guar gum and mannitol; and HPMC shows nearly 100% drug release within one hour (FIGS. 10, F5 and F6) while tablet comprising co-processed material of instant invention shows sustained release of drug(up to 100% of drug release in 8 Hrs.) (FIG. 9). Dissolution profile of F7 matches with the release profile of Voveran SR (up to 75% of drug release in 8 Hrs.) (FIG. 10)

### EXAMPLE 7

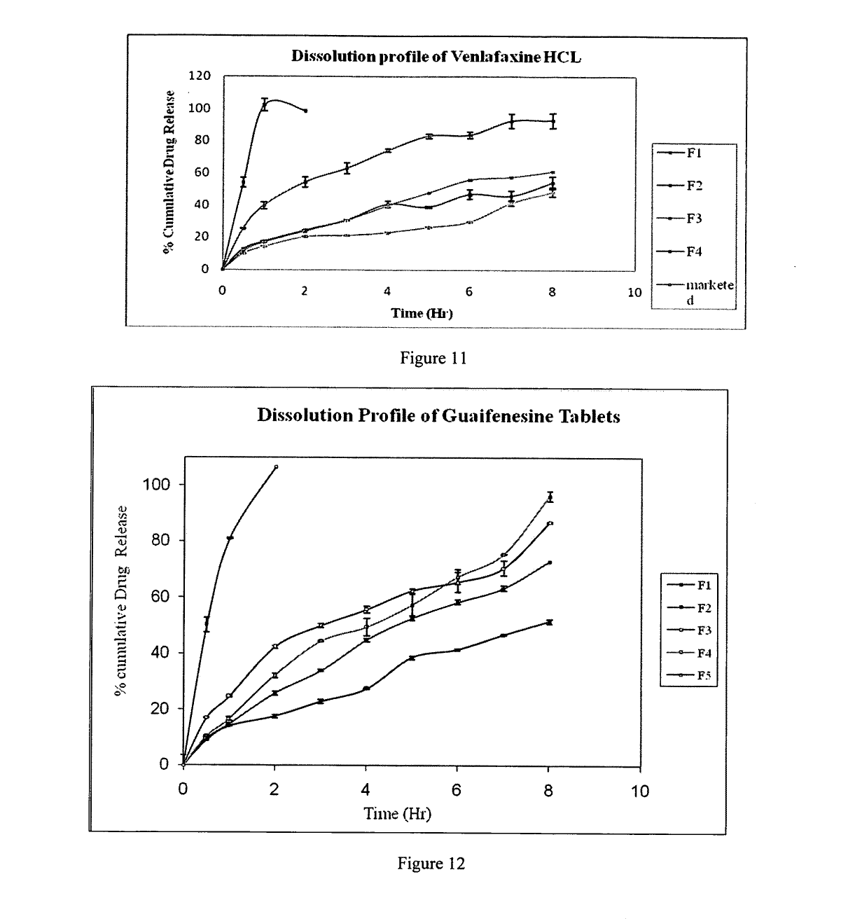

Tablets of venlafaxine HCL were prepared with physical mixture of guar gum and mannitol; HPMC and co-processed spray dried material, respectively, in the range of about 50% of the tablet weight (Table 3), tested for tabletting parameters and dissolution as represented herein in FIG. 11. The tablets prepared with co-processed material have sustained the delivery of the drug over 10 hours (F1).

TABLE 3
Formulation of Venlafaxine HCl

Quantity per Tablet in mg

| Ingredients | F1 | F2 | F3 | F4 |
|---|---|---|---|---|
| Drug | 84 | 84 | 84 | 84 |
| MCC 102 | 100 | 200 | 200 | 200 |
| HPMC K100M | — | — | 336 | — |
| SDGGMN 1 | 100 | 336 | — | — |
| PMGGMN1 | — | — | — | 336 |
| Mg Stearate | 3 | 6 | 6 | 6 |
| Talc | 6 | 12 | 12 | 12 |
| Total | 293 | 638 | 638 | 638 |
| % of Release | 34.12 | 52.66 | 52.66 | 52.66 |
| retardant material | | | | |

F—Formulations, MCC102—Microcrystalline Cellulose, HPMC K100M—Hydroxypropylmethylcellulose (1,00,000 cP), SDGGMN1—spray dried guar gum and mannitol(1:1), PMGGMN 1—physical mixture of guar gum and mannitol(1:1).

### EXAMPLE 8

Tablets of guaifenesin were prepared with physical mixture of guar gum and mannitol; HPMC and co-processed spray dried material, respectively, in the range of about 6-14% of the tablet weight (Table 4), tested for tabletting parameters and dissolution as represented herein in FIG. 12. The tablets prepared with co-processed spray dried material have sustained the delivery of the drug over 8 hours (F1, F2 and F4). Spray dried material added to the Drug granules show that co-processed material can be formulated with granules.

TABLE 4
Formulation of Guaifenesin

| | Quantity per Tablet in mg | | | | |
|---|---|---|---|---|---|
| Ingredients | F1 | F2 | F3 | F4 | F5 |
| Drug | 600 | 600 | 600 | 600 | 600 |
| MCC 101 | 80 | 80 | 80 | 80 | 80 |
| PVP k30 | 14 | 14 | 14 | 14 | 14 |
| HPMC K100M | — | — | 60 | — | — |
| SPGGMN 1 | 120 | 60 | — | 50 | — |
| PMGGMN 1 | — | — | — | — | 60 |
| Mg Stearate | 7 | 7 | 7 | 7 | 7 |
| Talc | 14 | 14 | 14 | 14 | 14 |
| Total | 835 | 775 | 775 | 765 | 775 |
| % of Release | 14.37 | 7.74 | 7.74 | 6.53 | 7.74 |
| retardant Material | | | | | |

F—Formulations, MCC101—Microcrystalline Cellulose, HPMC K100M—Hydroxypropylmethylcellulose (1,00,000 cP), SDGGMN1—spray dried guar gum and mannitol(1:1), PMGGMN 1—physical mixture of guar gum and mannitol(1:1), PVP K30—Poly vinyl pyrrolidone.

### EXAMPLE 9

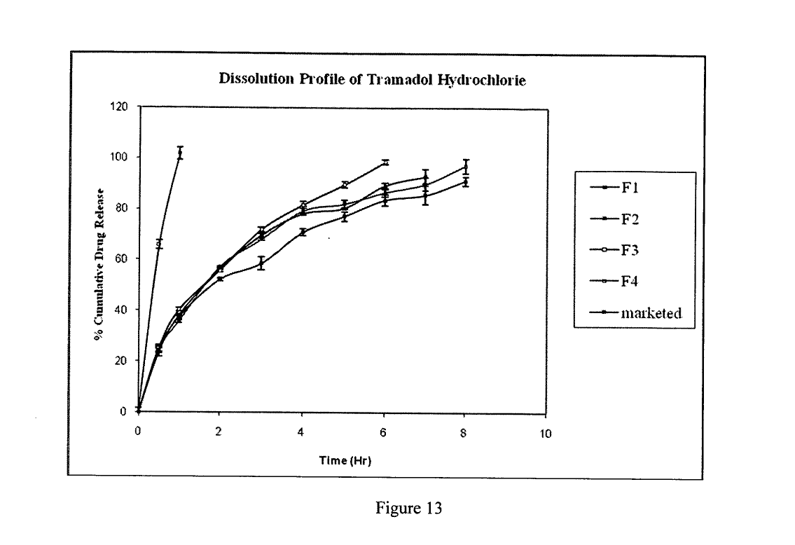

Tablets of tramodol were prepared with physical mixture of guar gum and mannitol; HPMC and co-processed spray dried material, respectively, in the range of about 52% of the tablet weight (Table 5), tested for tabletting parameters and dissolution as represented herein in 13. The tablets prepared with co-processed material have sustained the delivery of the drug over 8 hours (F2) comparable to marketed product. Tablet (F4) containing physical mixture released drug within one hour, see FIG. 13.

TABLE 5
Formulation of Tramadol HCl Tablets

Quantity per Tablet in mg

| Ingredients | F1 | F2 | F3 | F4 |
|---|---|---|---|---|
| Tramadol HCl | 100 | 100 | 100 | 100 |
| MCC 102 | 50 | 15 | 50 | 50 |
| HPMC K100M | — | — | 100 | — |
| SPGGMN 1 | 100 | 135 | — | — |
| PMGGMN1 | — | — | — | 100 |
| Mg Stearate | 2.5 | 2.5 | 2.5 | 2.5 |
| Talc | 5 | 5 | 5 | 5 |
| Total | 257.5 | 257.5 | 257.5 | 257.5 |
| % of release | 38.91 | 52.52 | 38.91 | 38.91 |
| retardant material; | | | | |

F—Formulations, MCC102—Microcrystalline Cellulose, HPMC K100M—Hydroxypropylmethylcellulose (1,00,000 cP), SDGGMN1—spray dried guar gum and mannitol(1:1), PMGGMN 1—physical mixture of guar gum and mannitol(1:1).

### EXAMPLE 10

Preparation of Sustained Released Diclofenac Sodium Tablets:

Spray Drying Guar Gum/Mannitol:

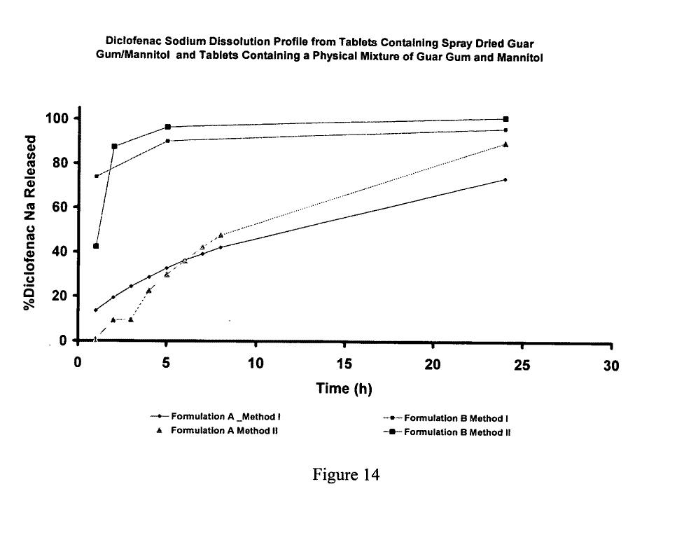

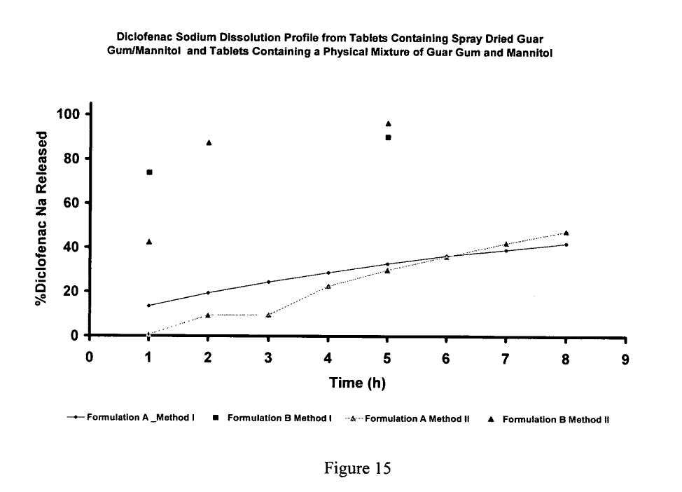

The solution/suspension was prepared by mixing 1.5 g mannitol with 1.5 g of guar and then blending with a Turrax homogenizer. This produced a 0.5% solution/suspension that would work in the spray drier. A higher concentration of 1% produced a solution/suspension that due to its high viscosity would not work in the spray drier system due to clogging of the nozzle. The air nozzle was used on the spray drier. The dryer was run at a 195° C. inlet temperature, with a pump rate of 3 ml/min and an air flow at 65 n/m². This gave a light yellow colored powder. This powder was then used in sustained release studies using diclofenac at a 16% loading level. The guar/mannitol spray dried material 0.5 g, was combined with 1.0 g of Ran Q MCC, and 1.0 g of diclofenac sodium. The tablets were pressed out a 3000 lb, at 500 mg each. Additionally; a similar mechanical blend was produced with 0.25 g guar gum. 0.25 g of mannitol, 1.0 g of Ran Q MCC, and 1.0 g of diclofenac sodium. Below in Table 8 are the detailed studies carried out with the diclofenac sodium tablets containing the sustained release spray dried material, shown in FIGS. 14 and 15.

Method I:

Dissolution medium: pH 6.8 Na phosphate buffer; 900 mL; 37±0.5° C.

Apparatus II (paddle): 50 rpm

Samples were withdrawn at each hour for 8 hours and then at 24 hours.

The amount of Diclofenac Na released was determined from the UV absorbance's at the wavelength of maximum absorbance at 276 nm on filtered portions of the solution/suspension under test in comparison with a standard solution/suspension prepared as recommended in the USP method for Diclofenac Sodium delayed-release tablets, buffer stage.

Method II: (Adaptation of the USP Method for Diclofenac Sodium Delayed-Release Tablets)

Acid Stage

Dissolution medium: 0.1N HCl; 900 mL; 37±0.5° C.

Apparatus II (paddle): 50 rpm

After 1 hour the HCl 0.1N was decanted from the dissolution vessel and the remaining of the tablet was subjected to the buffer stage (see below).

To the 0.1N HCl resulted from the dissolution were added 20 mL of NaOH 5N. The amount of Diclofenac Na released was determined from the UV absorbance at the wavelength of maximum absorbance at 276 nm on filtered portions of the solution/suspension under test in comparison with a standard solution/suspension prepared as recommended in the USP method for Diclofenac Sodium delayed-release tablets, acid stage.

Buffer Stage

Dissolution medium: pH 6.8 Na phosphate buffer; 900 mL; 37±0.5° C.

Apparatus II (paddle): 50 rpm

Samples were withdrawn at each hour for 7 hours and then at 24 hours.

The amount of Diclofenac Na released was determined from the UV absorbance at the wavelength of maximum absorbance at 276 nm on filtered portions of the solution/suspension under test in comparison with a standard solution/suspension prepared as recommended in the USP method for Diclofenac Sodium delayed-release tablets, buffer stage.

TABLE 6
Formulation A (using spray dried guar gum/mannitol)

| | Amount/ | | Amount/ |
|---|---|---|---|
| Ingredient | batch (mg) | % | tablet (mg) |
| Diclofenac Na | 2000 | 43.48 | 217.4 |
| Spray dried guar gum/mannitol | 600 | 13.04 | 65.2 |
| Microcrystalline cellulose | 2000 | 43.48 | 217.4 |
| Total | 4600 | 100 | 500 |

TABLE 7
Formulation B (using a physical
mixture of guar gum and mannitol)

| | Amount/ | | Amount/ |
|---|---|---|---|
| Ingredient | batch (mg) | % | tablet (mg) |
| Diclofenac Na | 2000 | 43.48 | 217.4 |
| Guar Gum | 300 | 6.52 | 32.60 |
| Mannitol | 300 | 6.52 | 32.60 |
| Microcrystalline cellulose | 2000 | 43.48 | 217.4 |
| Total | 4600 | 100 | 500 |

Wet Granulation of Guar/Mannitol

Guar gum 60 g, Mannitol 60 g, and water 25 g were wet granulated using the following conditions, low impeller 870 rpm, low chopper 1000 rpm, dry blending time 2 minutes, high impeller 700 rpm, high chopper 1500 rpm, water addition 16 rpm, wet massing time 1 min, dried to 3% LOD. This wet granulated material was used to produce test tablets pressed out of with acetaminophen at 16% loading, 500 mg of guar/mannitol, 1.2 g of RanQ MCC, and 0.320 g acetaminophen Compact PVC. Tablets of 500 mg were pressed at 3000 lb. These tablets were found to be unsuitable for a sustained release study since the tablets disintegrated in the medium in less than 30 seconds. The tablets that were produced from the sprayed dried material remained intact for over 24 hours. Comparison of the tablets of Example 10 and 11 are given in Table 8.

TABLE 8
RESULTS

| Sample | Time | % Diclofenac | Sample | Time | % Diclofenac |
|---|---|---|---|---|---|
| Name | (h) | Na Released | Name | (h) | Na Released |
| Formu- | 1 | 13.48 | Formu- | 1 | 73.81 |
| lation A | 2 | 19.39 | lation B | 2 | |
| Tablet | 3 | 24.43 | Tablet | 3 | |
| weight: | 4 | 28.68 | weight: | 4 | |
| 501.4 mg | 5 | 32.77 | 501.6 mg | 5 | 90.15 |
| Method I | 6 | 36.31 | Method I | 6 | |
| | 7 | 39.11 | | 7 | |
| | 8 | 42.09 | | 8 | |
| | 24 | 73.63 | | 24 | 96.15 |
| Formu- | 1 | 0.57* | Formu- | 1 | 42.36* |
| lation A | 2 | 9.34 | lation B | 2 | 87.44 |
| Tablet | 3 | 9.54 | Tablet | 3 | |
| weight: | 4 | 22.55 | weight: | 4 | |
| 504.5 mg | 5 | 29.86 | 500.6 mg | 5 | 96.49 |
| Method II | 6 | 36.00 | Method II | 6 | |
| | 7 | 42.18 | | 7 | |
| | 8 | 47.55 | | 8 | |
| | 24 | 89.74 | | 24 | 101.10 |

*Acid Stage

### EXAMPLE 11

Sustained Release Acetaminophen Tablets:

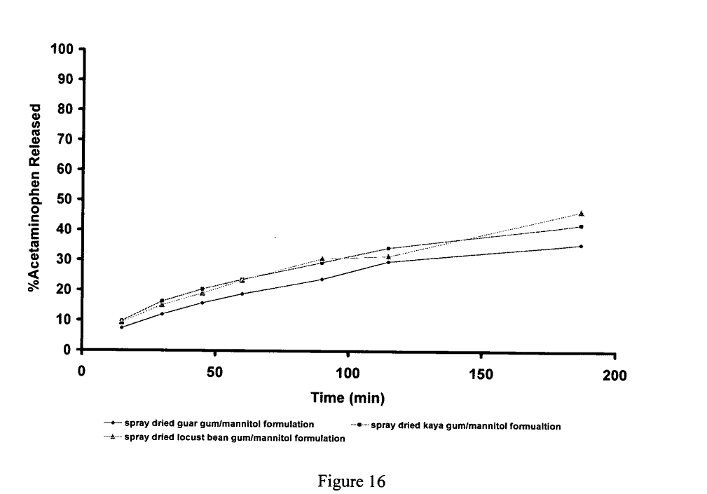

Sustained release acetaminophen tablets were prepared and test according to Example 10 with acetaminophen at 16% loading, 500 mg of guar/mannitol, 1.2 g of RanQ MCC, and 0.320 g acetaminophen Compact PVC. Tablets of 500 mg were pressed at 3000 lb. The results are illustrated in FIG. 16. All dissolutions with the acetaminophen were carried using the method below:

Dissolution medium: 0.1N HCl; 900 mL; 37±0.5° C.

Apparatus II (paddle): 50 rpm

### EXAMPLE 12

Spray Drying Loctus Bean/Mannitol:

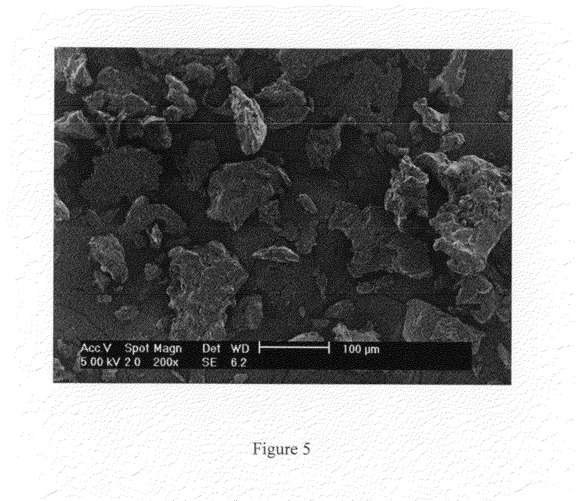

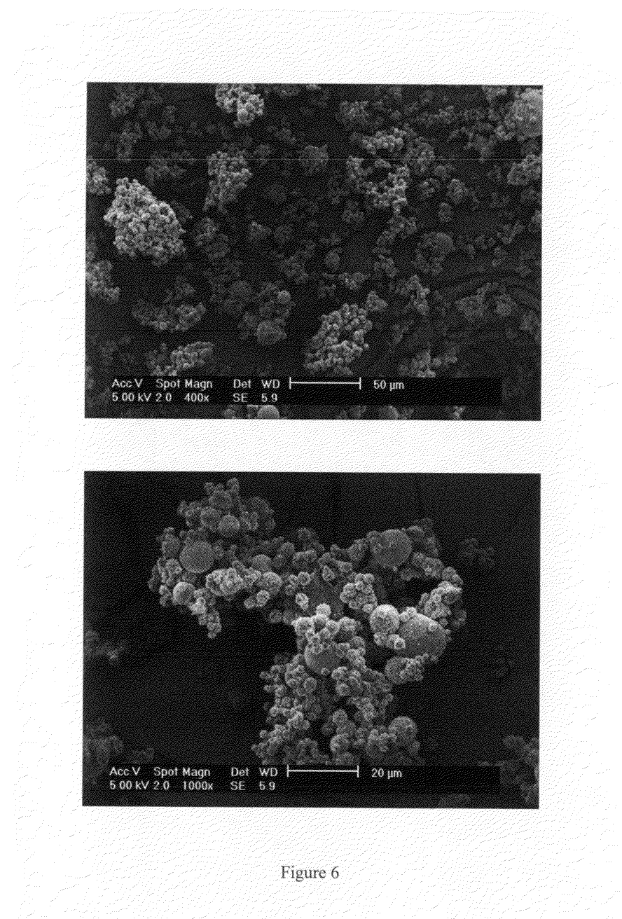

The solution/suspension was prepared by mixing 6 g mannitol, with 6 g of loctus bean and then blending with a Turrax homogenizer. This produced a 2% solution/suspension that would work in the spray drier. The drier was run at a 195° C. inlet temperature, with a pump rate of 3 ml/min and an air flow at 65 n/m2. This gave a white powder. The powder was tested for sustained release with acetaminophen. Experiments were the run with acetaminophen at 16% loading, 500 mg of loctus bean/mannitol, 1.2 g of RanQ MCC, and 0.320 g acetaminophen Compact PVC. SEM micrographs of the plain locust bean gum and the spray dried product according to this Example are shown at FIGS. 5 and 6 respectively.

### EXAMPLE 13

Spray Drying Karaya Gum/Mannitol:

The solution/suspension was prepared by mixing 6 g mannitol, with 6 g of Karaya gum and then blending with a Turrax homogenizer. This produced a 2% solution/suspension that would work in the spray drier. The drier was run at a 195° C. inlet temperature, with a pump rate of 3 ml/min and an air flow at 65 n/m2. This gave a white powder. The powder was tested for sustained release with acetaminophen. Experiments were the run with acetaminophen at 16% loading, 500 mg of Karaya gum/mannitol, 1.2 g of RanQ MCC, and 0.320 g acetaminophen Compact PVC.

The products described in Examples 14 through 16 were produced in a Sono-Tek laboratory spray drier, equipped with an air spray nozzle. The spray drying was done using an air inlet temperature of 190° C., air flow of 70 N/m², and a pump flow rate of 3 ml/min. The samples were prepared by dissolving the polyhydric sugar alcohol or the oligosaccharide in water by using a high speed rotary homogenizer. The polysaccharide gum was subsequently introduced slowly to the above prepared solution to ensure complete wetting. The whole mixture was then homogenized for 5 minutes. The mixture was then transferred over to the spray drier and kept under constant stirring with a magnetic stirrer throughout the spray drying process.

### EXAMPLE 14

Preparation of Guar Gum/Mannitol 1:4 Spray Dried Material

Mannitol 24 g (Roquette, Pearlitol 160 C) was homogenized in 1200 mL of deionized water. Guar gum 6 g (Coyote Brand, HV) was slowly added to the mixture while undergoing homogenization. The mixture was then spray dried to produce the Guar gum/mannitol material. The controlled release ability of the resulting material was tested by taking 300 mg of the guar gum/mannitol product and blending the material with 1.0 g of microcrystalline cellulose, and 1.0 g of sodium diclofenac. 500 mg tablets with a 13 mm diameter were pressed using a Carver manual press and a compression force of 3000 lbs. The tablets were then tested for dissolution using USP Apparatus II (paddle) at 50 rpm and 900 mL dissolution medium at 37±0.5° C. The dissolution experiment was performed in two stages: acid stage (dissolution medium HCl 0.1) for the first two hours and buffer stage (pH 6.8, 0.05 M sodium phosphate buffer) from 2 to 24 hours. After 7 hours 43% of the API is released and after 24 hours 94% of the API is released. Similar studies with 1:1 guar gum:mannitol ratio show 42% API release in 7 hours.

### EXAMPLE 15

Preparation of Cold Water Soluble Locust Bean Gum/Mannitol 1:1 Spray Dried Material

Mannitol 18 g (Roquette, Pearlitol 160 C) is homogenized in 1200 mL of deionized water. Cold water soluble loctus bean gum 18 g (Pangaea, Cold Water Soluble Locust Bean Gum) was slowly added to the mixture while undergoing homogenization. The mixture was then spray dried to produce the Locust Bean Gum/mannitol material. The sustained release ability of the resulted material was tested by taking 300 mg of the locust bean gum/mannitol product and blending the material with 1.0 g of microcrystalline cellulose, and 1.0 g of sodium diclofenac. 500 mg tablets with a 13 mm diameter were pressed using a Carver manual press and a compression force of 3000 lbs. The tablets were then tested for dissolution using USP Apparatus II (paddle) at 50 rpm and 900 mL dissolution medium at 37±0.5° C. The dissolution experiment was performed in two stages: acid stage (dissolution medium HCl 0.1) for the first two hours and buffer stage (pH 6.8, 0.05 M sodium phosphate buffer) from 2 to 24 hours. After 3 hours 5.4% API was released, after 7 hours 17% of the API was released and after 24 hours 57% of the API was released. Similar tablets using only locus bean gum and MCC show over 72% API release in 3 hours. The cold water soluble locust bean gum is not highly gelling material. The effective retardation of API release is surprising and indicates uniqueness of the composition produced by this invention

### EXAMPLE 16

Preparation of Guar Gum/Inulin 1:1 Spray Dried Material:

Inulin 6 g (Orafti ST-Gel) is homogenized in 1200 mL of deionized water. Guar gum 6 g (Coyote Brand, HV) was slowly added to the mixture while undergoing homogenization. The mixture was then spray dried to produce the Guar gum/inulin material. The sustained release ability of the resulted material was tested by taking 300 mg of the guar gum/inulin product and blending the material with 1.0 g of microcrystalline cellulose, and 1.0 g of sodium diclofenac. 500 mg tablets with a 13 mm diameter were pressed using a Carver manual press and a compression force of 3000 lbs. The tablets were then tested for dissolution using USP Apparatus II (paddle) at 50 rpm and 900 mL dissolution medium at 37±0.5° C. The dissolution experiment was performed in two stages: acid stage (dissolution medium HCl 0.1) for the first two hours and buffer stage (pH 6.8, 0.05 M sodium phosphate buffer) from 2 to 24 hours. After 7 hours 16% of the API is released and after 24 hours 47% of the API is released. Similar mannitol based composition (Guar Gum:mannitol, 1:1) show about 19% API release after 7 hours and 61% API released after 24 hours. This example indicates further retardation of API release by using inulin type molecule. Similar material made with Guar Gum: Mannitol and Guar Gum:Inulin at 1:4 ratio show 94% and 47% release respective. This indicates further retardation by inulin.

All percentages used herein are wt/wt percentages unless otherwise noted.

Having described the invention in detail, those skilled in the art will appreciate that modifications may be made of the invention without departing from its' spirit and scope. Therefore, it is not intended that the scope of the invention be limited to the specific embodiments described. Rather, it is intended that the appended claims and their equivalents determine the scope of the invention.

1. A sustained release composition comprising substantially spherical particles of at least one polysaccharide gum in combination with at least one polyhydric sugar alcohol.

2. A sustained release composition comprising a spray dried mixture of at least one polysaccharide gum in combination with at least one polyhydric sugar alcohol.

3. The composition of claim 2 wherein the at least one polysaccharide gum is selected from the group consisting of guar gum, xanthan gum, locust bean gum, karaya gum, tara gum, Konjac gum and mixtures thereof; and the at least one polyhydric sugar alcohol is selected from the group consisting of mannitol, xylitol, maltitol, lactitol, sorbitol, erythritol, isomalt and mixtures thereof.

4. The composition of claim 2 wherein the at least one polysaccharide gum is guar gum and the at least one sugar alcohol is mannitol.

5. The composition of claim 2 comprising a ratio of the at least one polysaccharide gum to the at least one polyhydric sugar alcohol is about 1:05 to about 1:10.

6. The composition of claim 2 comprising a ratio of the at least one polysaccharide gum to the at least one polyhydric sugar alcohol is about 1:1 to about 1:3.

7. A method for producing a sustained release composition, the method comprising mixing at least one polysaccharide gum and at least one polyhydric sugar alcohol in a solvent to form a solution/suspension, and spray drying the solution/suspension to form particles of the sustained release composition.

8. The method of claim 7 wherein the at least one polysaccharide gum is selected from the group consisting of guar gum, xanthan gum, locust bean gum, karaya gum, tara gum, Konjac gum and mixtures thereof; and the at least one polyhydric sugar alcohol is selected from the group consisting of mannitol, xylitol, maltitol, lactitol, sorbitol, erythritol, isomalt and mixtures thereof.

9. The method of claim 7 wherein the at least one polysaccharide gum is guar gum and the at least one sugar alcohol is mannitol.

10. A method of making a sustained release pharmaceutical solid dosage form, the method comprising:
mixing at least one polysaccharide gum and at least one polyhydric sugar alcohol in a solvent to form a solution/suspension;
spray drying the solution/suspension to form particles of a sustained release composition;
mixing the sustained release composition with at least one filler and at least on active pharmaceutical ingredient to form a tabletting mixture; and
compressing the tabletting mixture to form the sustained release pharmaceutical dosage form.

11. The method of claim 10 wherein the at least one polysaccharide gum is selected from the group consisting of guar gum, xanthan gum, locust bean gum, karaya gum, tara gum, Konjac gum and mixtures thereof; and the at least one polyhydric sugar alcohol is selected from the group consisting of mannitol, xylitol, maltitol, lactitol, sorbitol, erythritol, isomalt and mixtures thereof.

12. The method of claim 10 wherein the at least one active pharmaceutical ingredient is mixed with the sustained release composition by wet granulation.

13. The method of claim 10 wherein the at least one polysaccharide gum is guar gum and the at least one sugar alcohol is mannitol.

14. A sustained release pharmaceutical solid dosage form comprising:
a spray dried mixture of at least one polysaccharide gum in combination with at least one polyhydric sugar alcohol;
at least one filler; and
at least one active pharmaceutical ingredient.

15. The solid dosage form of claim 14 wherein the at least one polysaccharide gum is selected from the group consisting of guar gum, xanthan gum, locust bean gum, karaya gum, tara gum, Konjac gum and mixtures thereof; and the at least one polyhydric sugar alcohol is selected from the group consisting of mannitol, xylitol, maltitol, lactitol, sorbitol, erythritol, isomalt and mixtures thereof.

16. The solid dosage form of claim 14 wherein the at least one polysaccharide gum is guar gum and the at least one sugar alcohol is mannitol.

17. The solid dosage form of claim 14 wherein the filler is selected from the group consisting of MCC, lactose, dicalcium phosphate and mixtures thereof.

18. A sustained release composition comprising a spray dried mixture of at least one polysaccharide gum in combination with at least one oligosaccharide.

19. The sustained release composition of claim 18 wherein the at least one oligosaccharide is inulin.

20. The sustained release composition of claim 18 further comprising at least one polyhydric sugar alcohol.

21. A method for producing the sustained release composition according to claim 18, the method comprising mixing the least one polysaccharide gum and the least one oligosaccharide in a solvent to form a solution/suspension, and spray drying the solution/suspension to form particles of the sustained release composition.

22. The method of claim 21 further comprising:
mixing the sustained release composition with at least one filler and at least on active pharmaceutical ingredient to form a tabletting mixture; and
compressing the tabletting mixture to form the sustained release pharmaceutical dosage form.

23. A sustained release pharmaceutical solid dosage form comprising:
a spray dried mixture of at least one polysaccharide gum in combination with at least one oligosaccharide;
at least one filler; and
at least one active pharmaceutical ingredient.
